(19) United States
(12) Patent Application Publication
(10) Pub. No.: US 2015/0342561 A1
(43) Pub. Date: Dec. 3, 2015

(54) ULTRASOUND DIAGNOSIS APPARATUS

(71) Applicant: KONICA MINOLTA, INC., Tokyo (JP)

(72) Inventor: Yoshihiro TAKEDA, Tokyo (JP)

(73) Assignee: KONICA MINOLTA, INC., Tokyo (JP)

(21) Appl. No.: 14/704,102

(22) Filed: May 5, 2015

(30) Foreign Application Priority Data
May 30, 2014 (JP) 2014-112487

(51) Int. Cl. A61B 8/08; A61B 17/34; A61B 8/00; A61B 8/14

(52) U.S. Cl. CPC A61B 8/0841; A61B 8/14; A61B 8/5207; A61B 17/3403; A61B 8/54; A61B 8/461; A61B 2017/3413

(57) ABSTRACT

An ultrasound diagnosis apparatus can accurately identify a tip position of a puncture needle. The apparatus includes: a transmitter/receiver that transmits at least one ultrasound to a subject, and receives a reflected wave of the ultrasound from the subject; a processor that processes data on the received reflected wave to create at least one image of a two-dimensional structure including a depth direction of the subject; a display controller that controls a display to display the created image of the two-dimensional structure; a position acquirer that acquires an estimated tip position of a puncture needle to be stuck into the subject; and a transmission controller that adjusts at least one parameter of a focal length, aperture size and sound-ray density of the ultrasound depending on the acquired estimated tip position, and controls the transmitter/receiver to transmit the ultrasound.

### BACKGROUND OF THE INVENTION

1. Field of the Invention

The present invention relates to an ultrasound diagnosis apparatus.

2. Description of Related Art

Traditional ultrasound diagnosis apparatuses inspect the interiors of subjects through emitting ultrasounds into the subjects to receive reflected waves (echoes) from the subjects, and conducting predetermined processes to signal data on the received waves. Such ultrasound diagnosis is used for various purposes, such as medical tests and treatments and inspection of the internal structures of buildings.

The use of the ultrasound diagnosis apparatuses is not limited to display of images based on the processed data on obtained reflected waves. The ultrasound diagnosis apparatuses are also used to identify and visualize the position of a puncture needle relative to a specific portion (target) in a subject during the sticking of the puncture needle into the target for sampling the target, discharging water from the target, or injecting or indwelling an agent or marker into the target. Such ultrasound image can facilitate rapid, certain, and ready treatment for the target in the subject.

A typical puncture needle is as fine as the resolution used in an ultrasound diagnosis, and is often diagonally stuck into a subject. Unfortunately, such a fine puncture needle cannot readily return reflected waves to an ultrasound probe which detects the reflected waves of the ultrasounds. The apparatus thus cannot identify the position of the puncture needle relative to the position to be punctured. In order to solve the problem, Japanese Patent Application Laid-Open Publication No. 2006-320378 discloses a technique involving emission of ultrasounds perpendicularly to the direction of a puncture needle, which can more certainly identify the position of the puncture needle on the basis of the reflected waves received from the puncture needle.

The typical puncture needle is tapered toward the tip having a width (diameter) close to the wavelength of ultrasounds, and has an appropriate shape depending on the operations and the properties of a specific portion. Accordingly, the ultrasounds emitted perpendicularly to the extending direction of the puncture needle may be scattered or diffracted at the tip of the puncture needle. This may reduce the reflection efficiency. Furthermore, the speed and direction of the puncture needle can vary depending on a manipulation of a puncturer (e.g., doctor) and a behavior of the subject. Such variations may cause inaccurate emission of ultrasounds to the tip of the puncture needle, resulting in no echoes.

### SUMMARY OF THE INVENTION

An object of the invention is to provide an ultrasound diagnosis apparatus that can accurately identify the position of the tip of a puncture needle.

To achieve at least one of the above objects, an ultrasound diagnosis apparatus in which one aspect of the present invention is reflected includes: a transmitter/receiver that transmits at least one ultrasound to a subject, and receives a reflected wave of the ultrasound from the subject; a processor that processes data on the received reflected wave to create at least one image of a two-dimensional structure including a depth direction of the subject; a display controller that controls a display to display the created image of the two-dimensional structure; a position acquirer that acquires an estimated tip position of a puncture needle to be stuck into the subject; and a transmission controller that adjusts at least one parameter of a focal length, aperture size and sound-ray density of the ultrasound depending on the acquired estimated tip position, and controls the transmitter/receiver to transmit the ultrasound.

An ultrasound diagnosis apparatus in which one aspect of the present invention is reflected includes: a transmitter/receiver that transmits an ultrasound to a subject, and receives a reflected wave of the ultrasound from the subject; a processor that processes data on the received reflected wave to create an image of a two-dimensional structure including a depth direction of the subject; a frequency setter that sets at least one of a frequency band of the ultrasound used for creating the image of the two-dimensional structure and an intensity distribution in the frequency band; a display controller that controls a display to display the created image of the two-dimensional structure; and a position acquirer that acquires an estimated tip position of a puncture needle to be stuck into the subject, wherein the frequency setter executes at least one of settings: to shift the frequency band used for creating the image of the two-dimensional structure to a lower frequency side within a predetermined range including the acquired estimated tip position in comparison with the frequency band in an external area of the predetermined range; and to shift the intensity distribution to a lower frequency side of the frequency band within the predetermined range in comparison with the intensity distribution in the external area.

Preferably, in the ultrasound diagnosis apparatus, the transmission controller displaces a focal position of the transmitted ultrasound from the estimated tip position.

Preferably, in the ultrasound diagnosis apparatus, the processor creates the image of the two-dimensional structure based on reflected waves of the ultrasounds transmitted in different directions from the transmitter/receiver to the subject, and the transmission controller controls a variation in the adjusted parameter of the ultrasound to be transmitted/received at a larger angle from a traveling direction of the puncture needle to be greater than a variation in the adjusted parameter of the ultrasound to be transmitted/received at a smaller angle from the traveling direction, in a plane of the two-dimensional structure.

Preferably, the ultrasound diagnosis apparatus further includes a frequency setter that sets at least one of a frequency band of the ultrasound used for creating the image of the two-dimensional structure and an intensity distribution in the frequency band, and the frequency setter executes at least one of settings: to shift the frequency band used for creating the image of the two-dimensional structure to a lower frequency side within a predetermined range including the acquired estimated tip position in comparison with the frequency band in an external area of the predetermined range; and to shift the intensity distribution to a lower frequency side of the frequency band within the predetermined range in comparison with the intensity distribution in the external area.

Preferably, in the ultrasound diagnosis apparatus, the display controller controls the display to display a predetermined indication when the transmitter/receiver transmits/receives the ultrasound whose parameter is adjusted by the transmission controller.

Preferably, in the ultrasound diagnosis apparatus, the position acquirer acquires the estimated tip position based on a difference among the multiple images of the two-dimensional structures acquired at different timings.

Preferably, in the ultrasound diagnosis apparatus, the position acquirer acquires the estimated tip position based on information on a traveling length and direction of the puncture needle, the information being obtained from a puncturing mechanism of the puncture needle.

### BRIEF DESCRIPTION OF THE DRAWINGS

The present invention will become more fully understood from the detailed description given hereinbelow and the appended drawings, and thus are not intended as a definition of the limits of the present invention, and wherein:

FIG. 1 illustrates the entire configuration of an ultrasound diagnosis apparatus according to an embodiment of the invention;

FIG. 2 is a block diagram illustrating the internal configuration of the ultrasound diagnosis apparatus;

FIG. 3 illustrates an example difference image created by an evaluation information generator;

FIG. 4 illustrates an example correlation map image created by the evaluation information generator;

FIG. 5 illustrates an example pixel-value variance image created by the evaluation information generator;

FIG. 6 illustrates an example differential-value variance image created by the evaluation information generator;

FIG. 7 illustrates the focal position of ultrasounds transmitted from the ultrasound diagnosis apparatus;

FIG. 8 illustrates the distribution of the sound-ray densities and frequencies of ultrasounds to be transmitted/received;

FIG. 9 is a flowchart illustrating the control procedure of a puncture-needle position estimation process;

FIG. 10 is a flowchart illustrating the control procedure of a puncture-needle imaging process;

FIG. 11A illustrates the focal position of transmission ultrasounds in an ultrasound diagnosis apparatus according to a second embodiment;

FIG. 11B illustrates the focal position of transmission ultrasounds in the ultrasound diagnosis apparatus according to the second embodiment;

FIG. 12 illustrates the depth of focus of ultrasounds to be transmitted;

FIG. 13 is a flowchart illustrating the control procedure of the puncture-needle imaging process executed in the ultrasound diagnosis apparatus according to the second embodiment; and

FIG. 14 illustrates the spatial distribution of ultrasounds to be transmitted/received.

### PREFERRED EMBODIMENT OF THE PRESENT INVENTION

Embodiments of the invention will now be described with reference to the accompanying drawings.

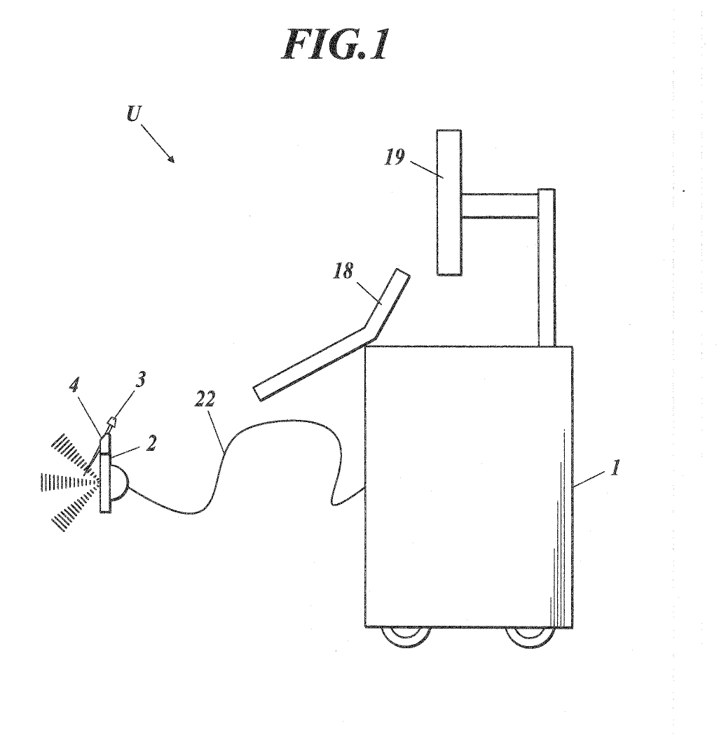

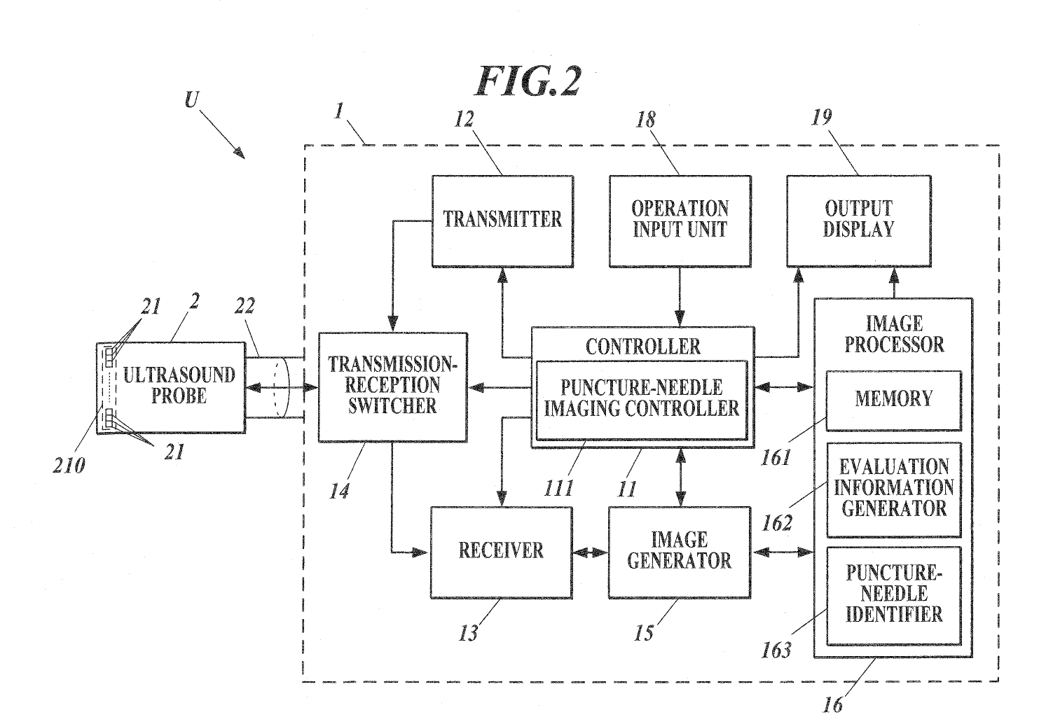

FIG. 1 illustrates the entire configuration of an ultrasound diagnosis apparatus U according to an embodiment. FIG. 2 is a block diagram illustrating the internal configuration of the ultrasound diagnosis apparatus U.

With reference to FIG. 1, the ultrasound diagnosis apparatus U includes an apparatus body 1, an ultrasound probe 2 (transmitter/receiver) connected to the apparatus body 1 via a cable 22, an attachment 4 (puncturing mechanism) mounted to the ultrasound probe 2, and a puncture needle 3.

The puncture needle 3 is elongated and hollow, and is configured to be stuck into a subject at an angle defined by the attachment 4. The puncture needle 3 may be replaced with another one having any appropriate thickness, length, and tip shape depending on a target (specimen) to be sampled and the type and volume of an agent to be injected.

The attachment 4 retains the puncture needle 3 in the set orientation (direction). The attachment 4 is mounted to a side of the ultrasound probe 2 and can appropriately change the orientation of the puncture needle 3 depending on a desired angle of the puncture needle 3 relative to the subject. The attachment 4 may include a measurer (not shown) for measuring a movement amount (travel length) of the puncture needle 3, and may transmit information on the measured movement amount and the orientation of the puncture needle 3 to the apparatus body 1 via the ultrasound probe 2 and the cable 22. Alternatively, the attachment 4 may transmit the information on the orientation of the puncture needle 3 alone to the apparatus body 1. The attachment 4 may have no means to transmit information on the puncture needle 3 to the apparatus body 1.

Alternatively, the ultrasound probe 2 may be directly provided with a guide section for retaining the puncture needle 3 in the puncture direction, in place of the attachment 4.

The apparatus body 1 includes an operation input unit 18 and an output display (display) 19. With reference to FIG. 2, the apparatus body 1 further includes a controller 11 (display controller), a transmitter 12, a receiver 13, a transmission-reception switcher 14, an image generator 15 (processor), and an image processor (position acquirer) 16.

The controller 11 of the apparatus body 1 outputs drive signals to the ultrasound probe 2 in response to an external operation on input devices, such as a keyboard and a mouse, of the operation input unit 18, and thus controls the ultrasound probe 2 to transmit ultrasounds. The controller 11 obtains reception signals generated in response to received ultrasounds from the ultrasound probe 2, executes various processes on the signals, and controls the output display 19 to display the results of the processes on a display screen as required.

The controller 11 includes a central processing unit (CPU), a hard disk drive (HDD), and a random access memory (RAM). The CPU reads various programs from the HDD and loads the programs in the RAM, to comprehensively control the operations of the individual components of the ultrasound diagnosis apparatus U under the instructions of the programs. The HDD stores a control program for operating the ultrasound diagnosis apparatus U, various processing programs, and various types of setting data. Alternatively, the programs and setting data may be rewritably stored in an auxiliary storage device including a non-volatile memory, such as a flash memory, instead of the HDD. The RAM is a volatile memory, for example, an SRAM or a DRAM. The RAM provides the CPU with a working memory space and stores temporary data.

The controller 11 includes a puncture-needle imaging controller 111 (transmission controller, frequency setter). The puncture-needle imaging controller 111 controls transmission/reception of ultrasounds on the basis of the positional information on the puncture needle 3 identified by the image processor 16 so as to capture a clear image of the tip of the puncture needle 3. The operation of the puncture-needle imaging controller 111 may be executed in the form of software using the CPU and the RAM of the controller 11.

The transmitter 12 outputs pulse signals to be supplied to the ultrasound probe 2 on the basis of control signals input from the controller 11, so that the ultrasound probe 2 generates ultrasounds. The transmitter 12 includes, for example, a clock generating circuit, a pulse generating circuit, a pulse width setter, and a delay circuit. The clock generating circuit generates clock signals for determining the transmission timing and frequency of pulse signals. The pulse generating circuit generates bipolar square-wave pulses having a predetermined voltage swing in predetermined cycles. The pulse width setter sets the width of the squire-wave pulse to be output from the pulse generating circuit. The square-wave pulses generated in the pulse generating circuit are distributed to wiring paths to individual oscillators 21 of the ultrasound probe 2, before or after the input to the pulse width setter. The delay circuit delays the timings of transmission of the generated square-wave pulses to the respective oscillators 21 by delay times set for the respective wiring paths.

The receiver 13 is a circuit that obtains reception signals input from the ultrasound probe 2 under the control of the controller 11. The receiver 13 includes, for example, an amplifier, an analog-digital conversion circuit, and a phasing addition circuit. The amplifier is a circuit that amplifies the reception signals generated in response to the ultrasounds received by the respective oscillators 21 of the ultrasound probe 2 by a predetermined amplification factor. The analog-digital conversion circuit converts the amplified reception signals into digital data at a predetermined sampling frequency. The phasing addition circuit adjusts the time phases of the digitized reception signals by adding a delay time to each of the signals for each of the wiring paths corresponding to the respective oscillators 21 and then adds these signals together, namely, performs phasing addition, to generate sound-ray data.

The transmission-reception switcher 14 performs switching between a mode of causing the transmitter 12 to output drive signals to the oscillators 21 and thus controlling the oscillators 21 to emit (transmit) ultrasounds, and a mode of causing the receiver 13 to output reception signals to receive signals related to the ultrasounds emitted from the oscillators 21, under the control of the controller 11.

The image generator 15 creates diagnostic images based on the received ultrasound data. The image generator 15 detects sound-ray data input from the receiver 13 through envelope detection to acquire signals. The image generator 15 may optionally execute logarithmic amplification, filtering (e.g., low-path filtering or smoothing), and/or enhancement on the acquired signals. The image generator 15 creates, as one of the diagnostic images, frame image data in a B-mode display which is represented by the luminance signals depending on the intensities of the acquired signals and shows the two-dimensional structure across the plane including the transmission direction (the depth in the subject) of the signals and the scanning direction of ultrasounds transmitted from the ultrasound probe 2. The image generator 15 may adjust the dynamic range for display and execute gamma correction. The image generator 15 may include a CPU and a RAM dedicated to the image creation. Alternatively, the image generator 15 may include a hardware configuration dedicated to the image creation on a substrate (e.g., application-specific integrated circuit (ASIC)). Alternatively, the image generator 15 may execute processes of the image creation using the CPU and the RAM of the controller 11.

The image processor 16 includes a memory 161, an evaluation information generator 162, and a puncture-needle identifier 163.

The memory 161 stores a predetermined number of frames of latest diagnostic image data (frame image data), which are processed by the image generator 15 to be used for real-time or substantially real-time display. The memory 161 is a volatile memory, such as a dynamic random access memory (DRAM). Alternatively, the memory 161 may be any high-speed rewritable non-volatile memory. The diagnostic image data stored in the memory 161 is read under the control of the controller 11, to be transmitted to the output display 19 or to be output to the outside of the ultrasound diagnosis apparatus U via a communication unit (not shown). If the output display 19 is in a television system, the scan format of the diagnostic image data should be converted by a digital signal converter (DSC) provided between the memory 161 and the output display 19 before the output.

The evaluation information generator 162 creates image data for the identification of the position of the puncture needle 3 by the puncture-needle identifier 163. The image data that can be created in the apparatus body 1 according to the embodiment will be described in detail below.

The evaluation information generator 162 can calculate, for example, the speed and traveling direction (movement vector) of the puncture needle 3 with reference to the history of the position of the puncture needle 3.

The puncture-needle identifier 163 identifies the current position of the puncture needle 3 through a process suitable for the image created by the evaluation information generator 162. The puncture-needle identifier 163 also calculates the estimated position of the puncture needle 3 at the subsequent time by applying the movement vector of the puncture needle 3 obtained by the evaluation information generator 162 with respect to the identified current position of the puncture needle 3. The information related to the calculated estimated position is output to the puncture-needle imaging controller 111 of the controller 11.

The evaluation information generator 162 and the puncture-needle identifier 163 may share a CPU and a RAM of the image processor 16, or may each include its own CPU and RAM. Alternatively, the evaluation information generator 162 and the puncture-needle identifier 163 may execute the processes using the CPU and the RAM of the controller 11.

The operation input unit 18 includes a push button switch, a keyboard, a mouse, a trackball, or any combination thereof. The operation input unit 18 converts operation inputs by the user into operation signals and inputs the signals to the apparatus body 1.

The output display 19 includes a display screen and a driving unit therefor. The display screen may use any display system, for example, a liquid crystal display (LCD), an organic electroluminescent (EL) display, an inorganic EL display, a plasma display, or a cathode-ray tube (CRT) display. The output display 19 generates drive signals for the display screen (individual display pixels) on the basis of the control signals output from the CPU of the image generator 15 and the image data processed by the image processor 16, and thus causes the display screen to display a menu and a status for ultrasound diagnosis and measured data based on the received ultrasounds. The output display 19 may also be equipped with an LED lamp indicating an ON/OFF state of the power.

The operation input unit 18 and the output display 19 may be integrated with a housing of the apparatus body 1, or may be connected to the apparatus body 1 from the outside thereof via a RGB cable, USB cable or HDMI (registered trademark) cable. In the apparatus body 1 equipped with an operation input terminal and a display output terminal, these terminals may be respectively connected to traditional peripheral devices for operation and display.

The ultrasound probe 2 oscillates ultrasounds (approximately 1 to 30 MHz in this embodiment) and emits the ultrasounds to the subject, such as a living body. The ultrasound probe 2 also functions as an acoustic sensor to receive the reflected waves (echoes) of the emitted ultrasounds that are reflected from the subject and to convert the echoes into electrical signals. The ultrasound probe 2 includes an oscillator array 210 composed of an array of multiple oscillators 21 for transmitting and receiving the ultrasounds, and a cable 22.

The cable 22 includes a connector (not shown) to the apparatus body 1 at an end, so as to removably connect the ultrasound probe 2 to the apparatus body 1. The user brings an ultrasound transmission/reception surface of the ultrasound probe 2, namely, a surface from which the oscillator array 210 emits the ultrasounds, into contact with the subject with a predetermined pressure, and activates the ultrasound diagnosis apparatus U for ultrasound diagnosis.

The oscillator array 210 is a one-dimensional array of multiple oscillators 21 in a predetermined direction (scanning direction), for example. The oscillators 21 each include a piezoelectric element including a piezoelectric body and electrodes that are provided at both ends of the piezoelectric body and are charged in response to a deformation (expansion or contraction) of the piezoelectric body. When the oscillators 21 are sequentially supplied with voltage pulses (pulse signals), the respective piezoelectric bodies deform in response to electric fields generated therein and emit ultrasounds. When the oscillators 21 receive ultrasounds in a predetermined frequency band, the acoustic pressure of the ultrasounds varies the thicknesses of the piezoelectric bodies (vibrates the piezoelectric bodies). This phenomenon generates electric charges corresponding to the variations, which charges are converted into electrical signals corresponding to the charge amount to be output.

An operation to detect and display the puncture needle 3 in the ultrasound diagnosis apparatus U according to the embodiment will now be explained.

The ultrasound diagnosis apparatus U according to the embodiment has a function, in a B-mode, to display a tomogram of the one-dimensional or two-dimensional structure in substantially real time on the basis of the luminance. The ultrasound diagnosis apparatus U also has a function, in a puncture-needle display mode, to more clearly display an image of the puncture needle 3 in the displaying status in the B-mode.

In the puncture-needle display mode in the ultrasound diagnosis apparatus U according to the embodiment, the image processor 16 identifies the position of the tip of the puncture needle 3 through the analysis of an image of the two-dimensional structure of the subject, which is captured as in a normal B-mode image. The image processor 16 also calculates the prediction position of the tip (estimated tip position) at the subsequent time on the basis of the history (direction and speed) of travel of the tip position. Then the operational setting for the transmission of ultrasounds to a predetermined range including the estimated tip position and/or the reception of ultrasounds reflected from the predetermined range are changed under the control of the puncture-needle imaging controller 111, so that echoes are readily received from the tip of the puncture needle 3.

The following explanation focuses on a process of identifying the tip position of the puncture needle 3. In the ultrasound diagnosis apparatus U according to the embodiment, the evaluation information generator 162 creates an image for identifying the tip position of the puncture needle 3 on the basis of differences among multiple frame images captured at different timings. The puncture-needle identifier 163 then identifies the tip position of the puncture needle 3 with reference to the image for identifying the tip position.

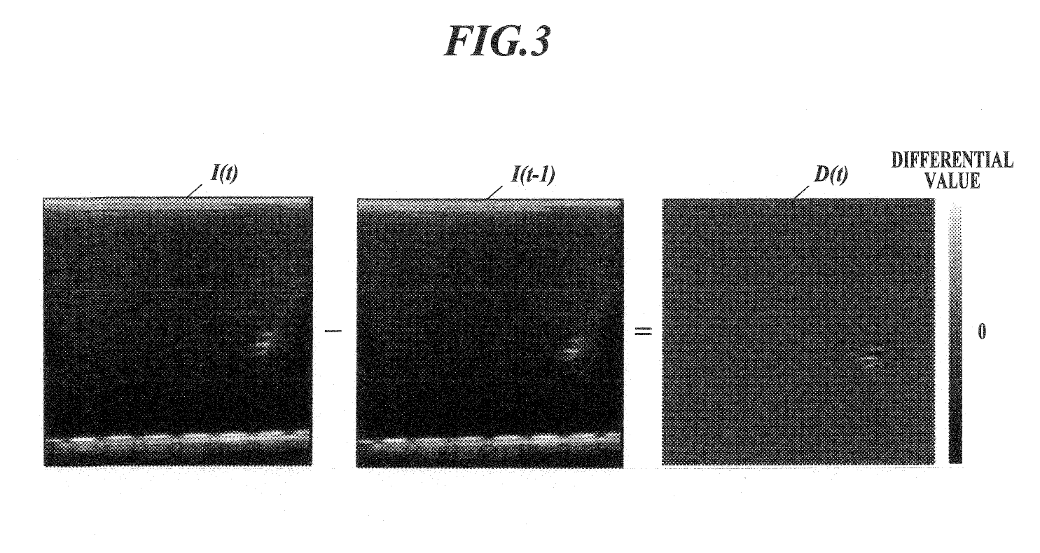

FIG. 3 illustrates an example difference image D(t) created by the evaluation information generator 162.

To create the difference image D(t), which is an example image for detecting the puncture needle 3 to be stucked, the evaluation information generator 162 calculates differences in pixel value between the pixels of an ultrasound image I(t) in the latest frame (timing T=t) and the pixels of an ultrasound image I(t−1) in the previous frame. During the sticking of the puncture needle 3 into the subject in a substantially static state, the movement of the puncture needle 3 varies the reflecting position of echoes particularly at or in the vicinity of the tip of the puncture needle 3 and thus causes a non-zero differential value in pixel value. The puncture-needle identifier 163 detects such a position having the non-zero differential value and thus identifies the tip position of the puncture needle 3.

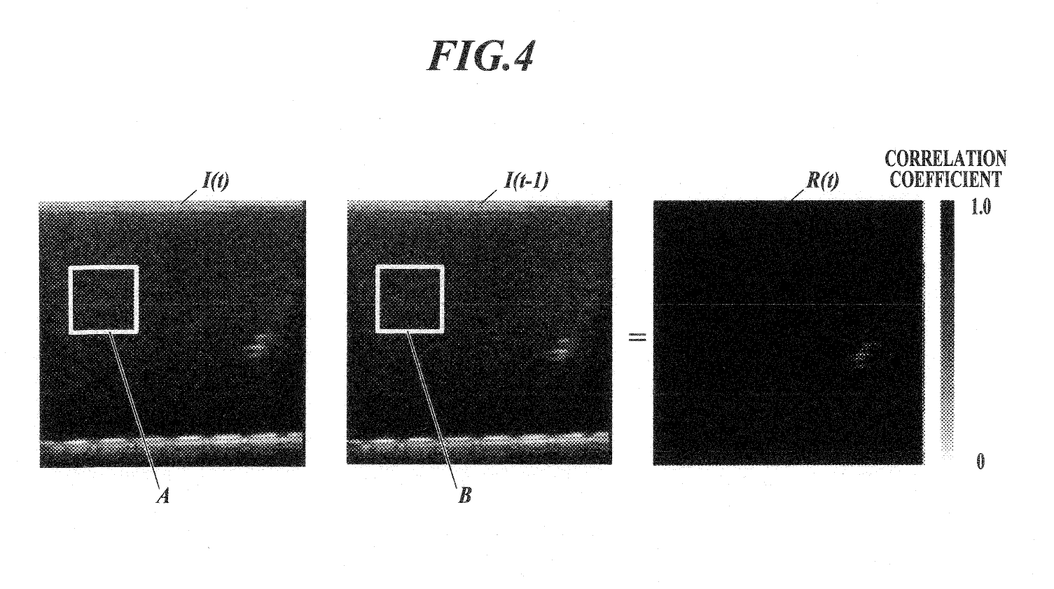

FIG. 4 illustrates an example correlation map image R(t) created by the evaluation information generator 162.

To create the correlation map image R(t), which is another example image for detecting the puncture needle 3, the evaluation information generator 162 determines the correlation among the pixel values of respective regions having a predetermined size and defined in multiple frames of ultrasound images. The resulting correlation map image R(t) shows the distribution of the correlation values. In specific, the evaluation information generator 162 defines, with respect to pixel coordinates (x,y) in the ultrasound images, a target region A(x,y,t) having the predetermined size around each pixel coordinate (x,y) in an ultrasound image I(t) in the latest frame (timing T=t), whereas defining a target region B(x,y,t−1) having the same size around each pixel coordinate (x,y) in an ultrasound image I(t−1) in the previous frame. The evaluation information generator 162 then calculates a correlation coefficient (cross-correlation coefficient) r (x,y,t) from each pixel value in the two regions. The resulting correlation map image R(t) shows the distribution of the calculated correlation coefficients r.

A variation in the ultrasound image between frames caused by the travel of the puncture needle 3 reduces the correlation coefficients r (x,y,t) in the regions. The correlation coefficients r do not readily increase (not readily approach 1) in small target regions A and B having pixel values deviated within a narrow range. Variations in the correlation coefficients r caused by the travel of the puncture needle 3 cannot be readily observed in numerical values in large target regions A and B. The size of the target regions A and B should thus be appropriately set.

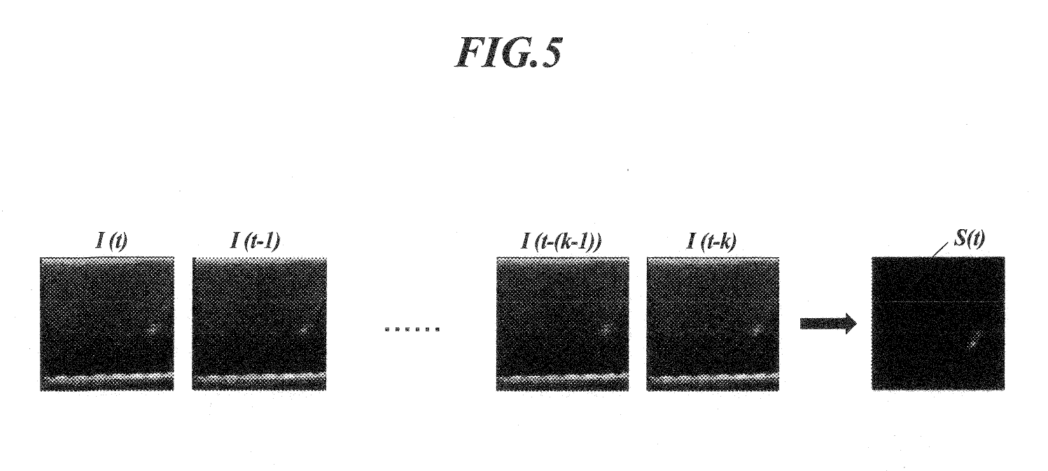

FIG. 5 illustrates an example pixel-value variance image S(t) created by the evaluation information generator 162.

To create the pixel-value variance image S(t), which is another example image for identifying the tip position of the puncture needle 3, the evaluation information generator 162 calculates a variance of pixel values among an ultrasound image I(t) in the latest frame (timing T=t) and previously captured k frames of ultrasound images I(t−1) to I(t−k) in each corresponding pixel. The evaluation information generator 162 may alternatively calculate a standard deviation instead of the variance. The variance increases in response to a temporary variation in pixel value (luminance) along the travel path of the puncture needle 3. As the movement amount across the k frames increases, a region having large variances expands in the pixel-value variance image S(t), so that the sticking speed and direction of the puncture needle 3 can be readily detected. The number k of frames used for the creation of the pixel-value variance image S(t) is appropriately set within the number of frames of ultrasound images stored in the memory 161. In particular, if the puncture needle 3 is stuck in the same direction, an increase in the number k of frames reduces the effects of noise and thus facilitates the detection of the puncture needle 3.

Alternatively, the number k of frames may be set depending on the frame rate of capturing ultrasound images. For example, the number FN of frames can be determined based on Expression (1):

FN=FPS/30×FNB (1)

where FPS indicates the frame rate, and FNB indicates the number of frames of ultrasound image data used for the calculation of variances at a frame rate of 30 fps.

In other words, since the ultrasound image data captured at a high frame rate have small differences between the frames, a large number of frames of ultrasound image data can improve the accuracy in the detection of the puncture needle 3. In contrast, since the ultrasound image data captured at a low frame rate have large differences between the frames, a small number of frames of ultrasound images can improve the throughput of the detection of the puncture needle 3.

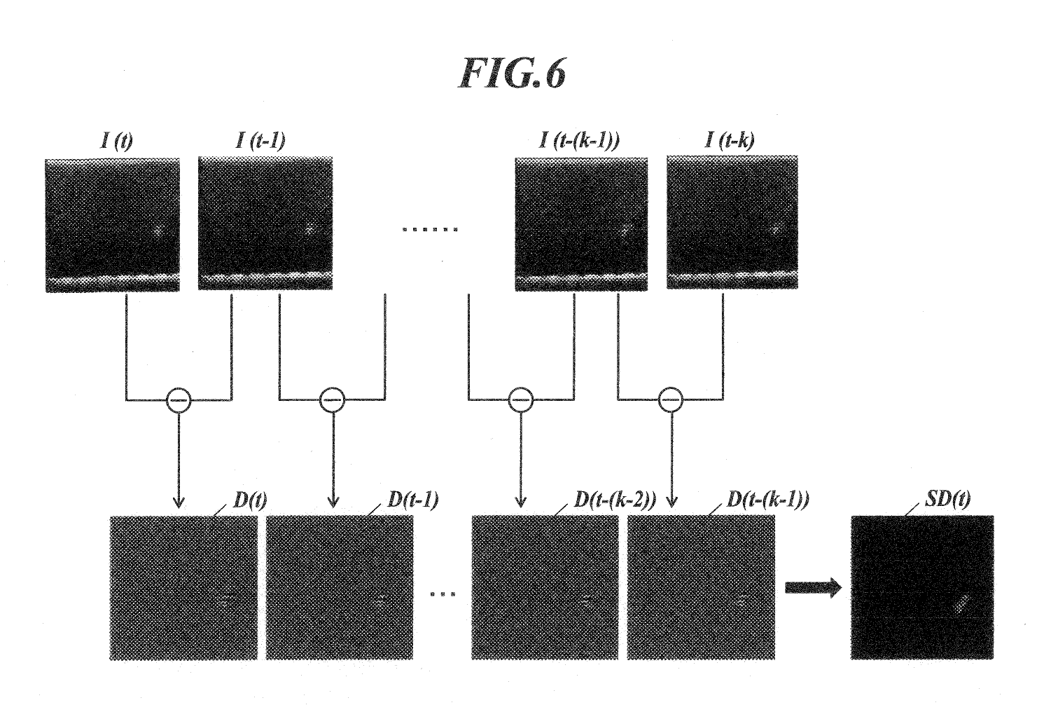

FIG. 6 illustrates an example differential-value variance image SD(t) created by the evaluation information generator 162.

To create the differential-value variance image SD(t), which is another example image for identifying the tip position of the puncture needle 3, the evaluation information generator 162 calculates differential values between each two adjacent frames as described with reference to FIG. 3 and then determines a variance of the differential values in each corresponding position, instead of calculating a variance of pixel values among multiple frames of ultrasound images in each corresponding position as described with reference to FIG. 5.

In specific, the evaluation information generator 162 creates a difference image D(t), showing differential values between the pixels of an ultrasound image I(t) in the latest frame and the pixels of an ultrasound image I(t−1) in the previous frame, to a difference image D (t−(k−1)), showing differences between the pixels of an ultrasound image I(t−(k−1)) ((k−1) frames before the latest frame) and the pixels of an ultrasound image I(t−k) (k frames before the latest frame). The evaluation information generator 162 then calculates a variance of the pixel values (differential values) among the multiple difference images in each corresponding pixel position, and thus creates a differential-value variance image SD(t).

At least one of the images for identifying the tip position of the puncture needle 3 illustrated in FIGS. 3 to 6 is created, and is used for the identification of the tip position of the puncture needle 3.

For the identification of the tip position of the puncture needle 3, the ultrasound diagnosis apparatus U may execute additional processes other than the above-described image processing. For example, the ultrasound diagnosis apparatus U may identify the tip position only within a predetermined area around the previously identified tip position. This control can eliminate the effects of an unexpected behavior or variation in the subject at a portion that is not expected to include the tip position, artifacts, and image noise, and can reduce the load of processes.

If the attachment 4 outputs information on the orientation and/or the movement amount of the puncture needle 3 to the apparatus body 1, the tip position of the puncture needle 3 may be calculated by using the information on the orientation and/or the movement amount of the puncture needle 3, in addition to or instead of identification of the tip position of the puncture needle 3 by the above-described processing of multiple images.

Alternatively, the traveling speed and direction (movement vector) of the tip position may be calculated based on the history of the tip position identified in the previous frames of ultrasound image data, and the current tip position of the puncture needle 3 may be identified only within a predetermined area around the position calculated by adding the displacement corresponding to the movement vector to the previously identified tip position. The starting point and orientation of the movement vector can be determined, for example, by calculating a primary regression line of the previously identified tip positions assuming a linear movement. The magnitude of the movement vector can be determined based on intervals between the identified tip positions or an average of the directional components of the calculated primary regression line.

In the ultrasound diagnosis apparatus U according to the embodiment, information on the current estimated position (estimated tip position) of the tip of the puncture needle 3 estimated by the puncture-needle identifier 163 is transmitted to the puncture-needle imaging controller 111 and is used to control the transmission/reception of ultrasounds for the capture of an ultrasound image in the subsequent frame.

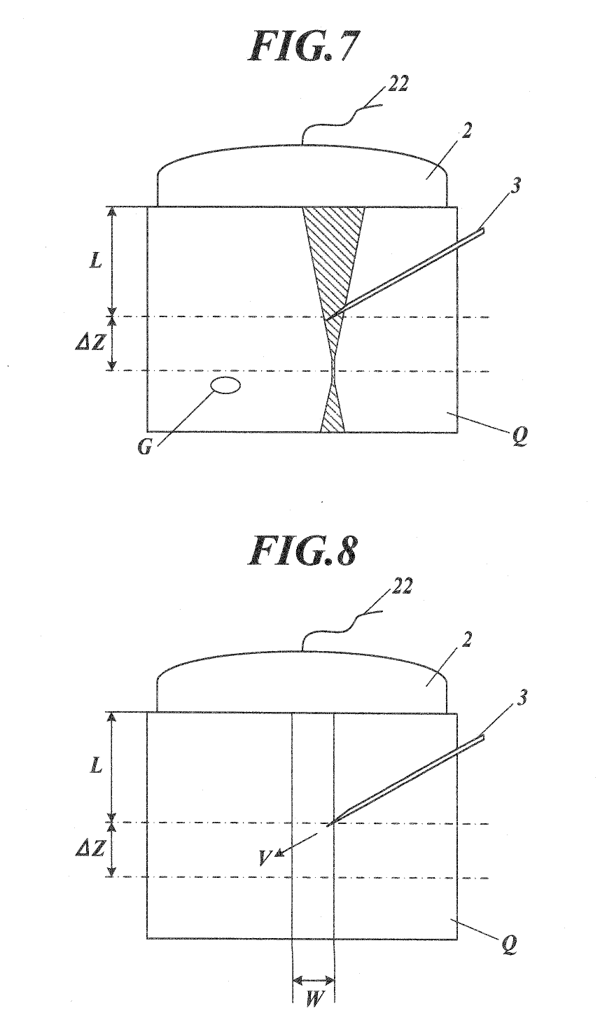

FIG. 7 illustrates the setting of the focus position of transmission ultrasounds by the ultrasound diagnosis apparatus U according to the embodiment.

In the ultrasound diagnosis apparatus U according to the embodiment, the focal position (focal point, focus position) of ultrasounds transmitted from the oscillators 21 of the ultrasound probe 2 is displaced by a predetermined distance from the estimated current tip position to a deeper position (i.e., a position more remote from the source of emission) in a subject Q, in other words, the focal length is increased. The ultrasound diagnosis apparatus U then creates a frame image for the display in the B-mode through the imaging of the two-dimensional structure of the subject Q. The focal position may be adjusted through any known technique, for example, the adjustment of a delay line or digital control signals for the transmitting/receiving timings of ultrasounds by the oscillators 21 in the capture of a single scanned image.

The puncture needle 3 is tapered toward the tip and is cut into an appropriate shape depending on the usage of the puncture needle 3 and the type of a target G, as described above. Upon the alignment of the focal point (focus) of transmission ultrasounds to the tip position of such a puncture needle 3 (at the depth L), even a small deviation of the focal position from the tip position of the puncture needle 3 in the plane perpendicular to the irradiation direction (beam axis) of ultrasounds often causes undesirable echoes. To address this problem, the ultrasound diagnosis apparatus U according to the embodiment sets the focal length of the transmission ultrasounds to be larger than the distance to the estimated tip position of the puncture needle 3 by a predetermined distance ΔZ. In other words, the transmission ultrasound beam is formed so as to have a large beam width (emission area) around the tip of the puncture needle 3, instead of being converged at the tip of the puncture needle 3, on the basis of the estimated tip position of the puncture needle 3. This adjustment ensures reception of echoes from the tip of the puncture needle 3. The focal position that is positioned deeper (more remote from the contact surface between the ultrasound probe 2 and the subject Q) than the tip position in the subject Q can be aligned to the target G, which usually resides deeper than the puncture needle 3, without excessive deviation.

The position of the target G at which the puncture needle 3 is stuck can be automatically detected or roughly set by the user using an electronic focusing scheme that can readily set multiple focal points. The ultrasounds can thus be transmitted and received with at least two focal points, i.e., the position displaced from the estimated tip position of the puncture needle 3 by the predetermined distance ΔZ and the position of the target G.

If the position of the target G is preliminarily determined as in the above case, the predetermined distance ΔZ can be varied depending on the distance or the difference in depth direction between the tip position of the puncture needle 3 and the position of the target G (position to be punctured). In specific, for the tip position of the puncture needle 3 remote from the target G, the predetermined distance ΔZ is set to an appropriate constant value for the clear display of the tip position. For the tip position of the puncture needle 3 close to the target G, the predetermined distance ΔZ can be varied within a range (within a predetermined range of the depth difference) that enables clear display of the tip position of the puncture needle 3, such that the focal point is aligned to or close to the target G for high-resolution display of the target G.

FIG. 8 illustrates the distribution of the sound-ray densities of transmission ultrasounds in the cross section along the scanning direction of the ultrasound probe 2.

In the normal display in B-mode, the subject Q is scanned with ultrasounds transmitted from the oscillators 21 in a predetermined direction (scanning direction), the ultrasounds having a uniform sound-ray density across the scanning positions or scanning direction. In specific, N oscillators 21 transmit/receive ultrasounds for the image creation in each scanning line while being shifted by M oscillators 21 between the adjacent scanning lines. In contrast, in the puncture-needle display mode, the sound-ray density of transmission ultrasounds is increased in a predetermined range W (first horizontal range) including the estimated tip position of the puncture needle 3. In specific, the N oscillators 21 are used for the image creation in each scanning line while being shifted by Mf oscillators 21 between the adjacent scanning lines, where Mf is smaller than M. This setting increases the number of ultrasound emissions per unit area while maintaining the area of a single emission of ultrasounds, and thus increases the total ultrasound emission area in the predetermined range W including the estimated tip position of the puncture needle 3.

In addition, the sound-ray density of transmission ultrasounds is decreased in an external area of the predetermined range W with the increase in sound-ray density in the predetermined range W. In specific, the N oscillators 21 are used for the image creation in each scanning line while being shifted by Mm oscillators 21 between the adjacent scanning lines, where Mm is larger than M.

An increase in the number of the scanning lines in the predetermined range W (caused by the shifting number Mf) should preferably be equal to a decrease in the number of the scanning lines in the external area of the predetermined range W (caused by the shifting number Mm), to maintain the total number of transmission/reception of data and the total amount of transmitted/received data in a single scanning operation. The ultrasound diagnosis apparatus U can thus display B-mode images at a high frame rate.

In the predetermined range W of an increased sound-ray density having a first (proximal) edge and a second (distal) edge, the width from the estimated tip position of the puncture needle 3 (or the currently identified tip position) to the second edge in the traveling direction of the tip of the puncture needle 3 (the puncture direction or the direction of a movement vector V) is preferably larger than the width from the estimated tip position to the first edge in the opposite direction. Forming a high-resolution image covering the larger width in the traveling direction of the puncture needle 3 (the left in FIG. 8) can efficiently provide a high-resolution image of the target to be sampled and the tip of the puncture needle 3. A high-resolution image of the tip of the puncture needle 3 with some margins can be obtained regardless of a certain variation in traveling speed of the puncture needle 3.

The predetermined range W of an increased sound-ray density can be varied depending on the speed (the magnitude |V| of the movement vector V) of the puncture needle 3 or the horizontal component (in the right-left direction in FIG. 8) of the speed. In other words, if the puncture needle 3 travels at a high speed |V|, a wide predetermined range W is set to prevent the deviation of the subsequent tip position of the puncture needle 3 from the predetermined range W. If the puncture needle 3 travels at a low speed |V|, a narrow predetermined range W is set to prevent a significant decrease in the image resolution of the external area of the predetermined range W. If the speed |V| is lower than a reference speed, the predetermined range W should be wider than a reference range corresponding to the reference speed. Such a variable predetermined range W can certainly include the tip of the puncture needle 3 and the target residing in the vicinity of the tip position, regardless of a future increase in the speed |V|.

Furthermore, the frequencies of ultrasounds to be transmitted/received in the predetermined range W can be varied, like the distribution of the sound-ray densities.

The oscillators 21 transmit the ultrasounds of continuous frequencies of a predetermined width (a certain frequency band). The oscillators 21 can be controlled to simultaneously output ultrasounds having different frequencies, for example, harmonics of a reference frequency. In general, a frequency compounding technique of transmitting ultrasounds having different frequencies to acquire echoes having different properties can compensate for speckles and an attenuation in the intensity of ultrasounds depending on their frequencies and thus clearly visualize a two-dimensional structure.

In the ultrasound diagnosis apparatus U according to the embodiment, the frequency band of ultrasounds to be transmitted/received is shifted to a lower frequency side in the predetermined range W in comparison with that in the external area. In specific, the ultrasound diagnosis apparatus U performs at least one of adjustments: to reduces the entire frequency band of ultrasounds to be transmitted/received; and to increase the ratio of low-frequency ultrasounds to the entire ultrasounds to be transmitted/received (i.e., shifts the distribution (intensity distribution) of the intensities of received ultrasounds to a lower frequency side of the reception frequency band). This operation can reduce the attenuation factor of ultrasounds during the propagation in the subject, so that the ultrasound diagnosis apparatus U can receive echoes with sufficient intensities from the vicinity of the tip of the puncture needle 3 deviated from the focal point. Such a regulation of the frequency band of received ultrasounds can be readily achieved with a known band transmission filter.

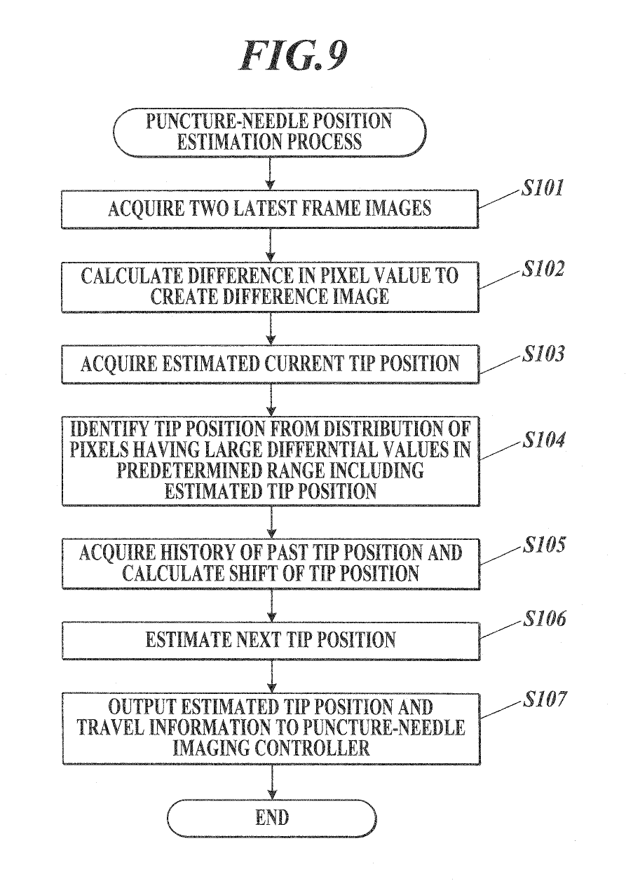

FIG. 9 is an example flowchart illustrating the control procedure of a puncture-needle position estimation process executed by a controller of the puncture-needle identifier 163 in the puncture-needle display mode according to the embodiment.

The puncture-needle position estimation process is executed after every creation of a predetermined number of new frame images in the image generator 15 and storage of the frame images in the memory 161 in the puncture-needle display mode, to estimate the position of the puncture needle 3 for the subsequent creation of a frame image. The following explanation focuses on the use of the difference image D(t) as an example image for identifying the tip position.

At the beginning of the puncture-needle position estimation process, the controller (CPU) of the puncture-needle identifier 163 acquires two latest frame images, i.e., the ultrasound image I(t) in the latest frame and the ultrasound image I(t−1) in the previous frame, from the memory 161 (Step S101). The controller calculates differences in pixel value between the respective pixels of the two ultrasound images to create a difference image D(t) (Step S102).

The controller acquires the estimated current tip position of the puncture needle 3, which was determined in Step S106 of the previous puncture-needle position estimation process (Step S103). The controller then detects pixels having large differential values in the predetermined range including the estimated tip position and identifies the current tip position on the basis of the distribution of the pixels having large differential values (Step S104).

The controller acquires the history of the tip position of the puncture needle 3 identified in every Step S104 of the previous puncture-needle position estimation processes, and then calculates a shift (i.e., the direction and speed) of the tip position including the current tip position (Step S105).

The controller estimates the tip position of the puncture needle 3 at the subsequent capture of a frame image on the basis of the current tip position of the puncture needle 3 and the calculated traveling direction and speed of the tip position of the puncture needle 3 (Step S106). The controller stores the estimated tip position and the information on the traveling direction and speed in a RAM of the controller, and outputs them to the puncture-needle imaging controller 111 (Step S107). The controller then terminates the puncture-needle position estimation process.

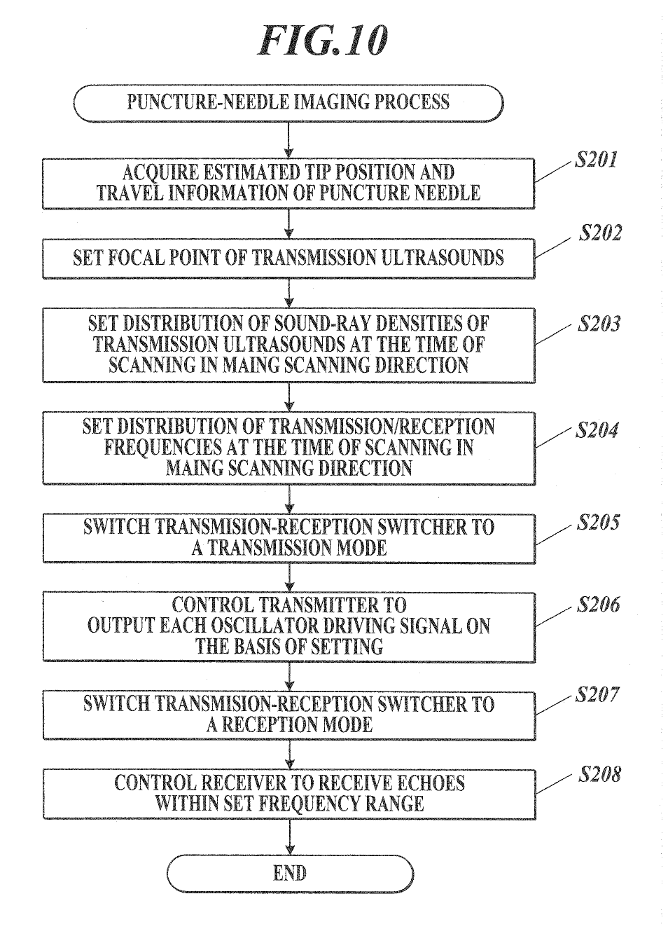

FIG. 10 is an example flowchart illustrating the control procedure of a puncture-needle imaging process executed by the puncture-needle imaging controller 111 in the puncture-needle display mode according to the embodiment.

The puncture-needle imaging process is executed every capture of a frame image.

The puncture-needle imaging controller 111 (CPU) acquires the estimated tip position of the puncture needle 3 and the traveling information (direction and speed) thereof input from the image processor 16 (Step S201).

The puncture-needle imaging controller 111 sets the focal position (i.e., the depth in the subject Q) of ultrasounds to be transmitted (Step S202). In specific, the puncture-needle imaging controller 111 displaces the focal position from the estimated tip position to a deeper position by a predetermined distance in the subject Q. The focal position thus shifts with time in response to a shift of the tip position of the puncture needle 3 every capture of a frame image.

The puncture-needle imaging controller 111 sets the distribution of the sound-ray densities of transmission ultrasounds at the time of scanning in the main scanning direction (Step S203). In specific, the puncture-needle imaging controller 111 defines a predetermined range W on the basis of the traveling direction of the tip position. The range W consists of a larger section, which is defined between the tip position and the second (distal) edge of the range W in the traveling direction of the tip position and has a predetermined width corresponding to the traveling speed of the tip position, and a smaller section, which is defined between the tip position and the first (proximal) edge of the range W in the opposite direction of the traveling direction. The puncture-needle imaging controller 111 then increases the sound-ray density in the predetermined range W and decreases the sound-ray density in an external area of the predetermined range W depending on the width of the predetermined range W.

The puncture-needle imaging controller 111 sets the distribution of the frequencies of transmission/reception ultrasounds at the time of scanning in the main scanning direction (Step S204). In specific, at the time of transmitting the ultrasounds, the puncture-needle imaging controller ill decreases the intensity of transmission waves in a high-frequency band (e.g., harmonics of the fundamental frequency) in the predetermined range W, and increases the intensity of transmission waves in a low-frequency band (e.g., at the fundamental frequency) correspondingly to the decrease. A the time of receiving ultrasounds by the receiver 13, the puncture-needle imaging controller 111 changes the band of the window of a filter for selectively introducing received ultrasounds in certain frequencies, such that the filter cuts reception signals in the high-frequency band in the predetermined range W.

The puncture-needle imaging controller 111 controls the transmission-reception switcher 14 to switch to a transmission mode (Step S205), and controls the transmitter 12 to output drive signals for the oscillators 21 on the basis of the above-described setting (Step S206). The puncture-needle imaging controller 111 then controls the transmission-reception switcher 14 to switch to a reception mode (Step S207), and controls the receiver 13 to receive echoes within the set frequency band (Step S208). The puncture-needle imaging controller 111 then terminates the puncture-needle imaging process.

After the image capture through the operations of the puncture-needle imaging process, the creation of a frame image in the image generator 15, and the storage of the frame image in the memory 161, the controller 11 controls the output display 19 to display the frame image on the display screen at an appropriate timing. The controller 11 can inform a user of the approximate position of the puncture needle 3, for example, with the display of a rectangular frame surrounding a predetermined area including the identified tip position of the puncture needle 3. The controller 11 may also provide the display of an arrow indicating the traveling direction of the puncture needle 3.

The controller 11 can provide a predetermined indication showing that the fame image of the two-dimensional structure on the display screen is displayed in the puncture-needle display mode instead of the normal displayed image in B mode. Examples of objects of the predetermined indication include a specific sign or mark representing the puncture-needle display mode. The controller 11 may also blink the indication object at predetermined time intervals or switch the color of the indication.

As described above, the ultrasound diagnosis apparatus U according to the embodiment includes the ultrasound probe 2 including an array of oscillators 21 for transmitting ultrasounds to the subject Q and receiving echoes thereof, the image generator 15 for processing signal data on the received echoes and creating an image of the two-dimensional structure of the subject Q along its depth direction, and the controller 11. The controller 11 controls the output display 19 to display the created image of the two-dimensional structure, and acquires the estimated tip position of the puncture needle 3 to be stuck into the subject Q from the puncture-needle identifier 163. The puncture-needle imaging controller ill then adjusts the total emission area of one or more ultrasound emission cycles from the oscillators 21 of the ultrasound probe 2 during the scanning operation in the predetermined range including the estimated tip position to be larger than the total ultrasound emission area of the target internal structure of the subject Q for the creation of a two-dimensional image. The controller 11 then controls the ultrasound probe 2 to transmit ultrasounds.

The ultrasound probe 2 can emit ultrasounds onto the fine tip, which may have any shape, of the puncture needle 3 and thus certainly receive echoes from the tip to accurately identify the tip position.

Such a displacement of the focal position of transmission ultrasounds from the estimated tip position of the puncture needle 3 by the puncture-needle imaging controller 111 prevents excess convergent ultrasound beams on the focal plane at the tip of the puncture needle 3. This control can thus prevent the deviation of the focal position of the convergent ultrasound beam from the tip of the puncture needle 3 and thus facilitate the reception of echoes.

The tip position of the puncture needle 3 is identified after every creation of a frame image in the image generator 15 and then the tip position at the subsequent capture of a frame image is estimated, and the focal length of the ultrasound beam is varied depending on a shift of the estimated tip position. This control can maintain an appropriate displacement of the focal point and thus prevent an excess reduction in the intensity of echoes caused by an excess displacement, leading to accurate detection and display of the tip of the puncture needle 3.

In particular, the difference in depth between the focal point of transmission ultrasounds and the estimated tip position in the subject Q is controlled within a predetermined range. This control can maintain the intensity of echoes from the tip of the puncture needle 3 at an appropriate level. The ultrasound diagnosis apparatus U can thus clearly display the tip of the puncture needle 3 with sufficient visibility regardless of a variation in the dynamic range, and can readily and accurately notify the user operating the puncture needle 3 of the tip position.

The predetermined distance ΔZ indicating the difference in depth between the focal point and the estimated tip position of the puncture needle 3 is determined based on the distance or the difference in depth in the subject Q between the tip position and the position to be punctured (the position of a target). The focal point can thus be shifted to an appropriate position in response to the approach of the tip of the puncture needle 3 to the target, so as to clearly display both the tip of the puncture needle 3 and the target. This control can increase the visibility of a displayed image showing the stuck puncture needle 3.

The focal point is positioned deeper than the estimated tip position of the puncture needle 3 in the subject Q. The focal point can thus be aligned to or close to the target to receive the puncture needle 3, which usually resides deeper than the estimated tip position of the puncture needle 3. This control can facilitate the clear display of the positional relationship between the target and the tip position of the puncture needle 3.

In a B-mode image of the two-dimensional structure, transmission ultrasounds in the predetermined horizontal range W including the estimated tip position have an increased sound-ray density than that in the external area of the predetermined range W. The fine tip of the puncture needle 3 can thus be certainly irradiated with the ultrasounds and more clearly displayed based on the echoes.

In particular, the predetermined range W has the second (distal) edge in the traveling direction of the puncture needle 3 and the first (proximal) edge in the opposite direction in the B-mode image of the two-dimensional structure. The predetermined range W has a larger width from the estimated tip position to the second edge than the width to the first edge. This control can facilitate the clear display of the tip of the puncture needle 3 and the target which is expected to be deviated from the tip position in the traveling direction. The control can also prevent the deviation of the tip of the puncture needle 3 from the clearly displayed area regardless of some variation in the traveling speed of the puncture needle 3.

The sound-ray density in the external area of the predetermined range W is reduced to decrease the number of ultrasound emissions in the external area, correspondingly to the increase in the number of ultrasound emissions caused by an increase in the sound-ray density in the predetermined range W. The total number of ultrasound emissions during a single scanning operation is thus constant. This configuration avoids the need for a variation in frame rate between the normal B-mode display and the puncture-needle display mode, and can thus simplify the controls. In addition, the configuration allows for a slight change in the displayed image between these modes, and can thus maintain high visibility to the user.

The controller 11 can set at least one of the frequency band of received ultrasounds used for the creation of a B-mode image of the two-dimensional structure, and the intensity distribution in the frequency band, to process data with the frequency compounding technique and create a frame image. The controller 11 then controls the output display 19 to display the frame image created through the process. The puncture-needle imaging controller 111 of the controller 11 acquires the estimated tip position of the puncture needle 3 to be stuck into the subject Q from the puncture-needle identifier 163. Before the subsequent capture of a frame image, the puncture-needle imaging controller 111 executes at least one of settings to shift the frequency band of received ultrasounds to a lower frequency side within the predetermined range W including the estimated tip position of the puncture needle 3 in comparison with the frequency band in the external area of the predetermined range W, and to shift the intensity distribution to a lower frequency side of the frequency band.

This control can compensate for speckles and an attenuation in the intensity of ultrasounds depending on their frequencies to clearly visualize the two-dimensional structure, and can accurately detect and display the tip of the puncture needle 3.

The puncture-needle identifier 163 identifies the position of the puncture needle 3 on the basis of the differences among multiple B-mode images based on echoes received at different timings, i.e., the difference image D(t), the correlation map image R(t), the pixel-value variance image S(t), or the differential-value variance image SD(t); and estimates the subsequent tip position on the basis of the history of travel of the identified puncture needle 3. The ultrasound diagnosis apparatus U thus requires no additional configuration for estimating the tip position of the puncture needle 3. The ultrasound diagnosis apparatus U can also provide a synergistic effect that the clear echoes from the tip of the puncture needle 3, which is achieved by the technique of transmitting ultrasounds according to the invention, lead to definite identification of the tip position.

The tip position of the puncture needle 3 may be estimated based on the information on the travel length and direction of the puncture needle 3 obtained by the operation of the attachment 4. The tip position of the puncture needle 3 can thus be certainly identified even if the tip position is misidentified in the image processor 16 due to noise or the like.

### Second Embodiment

The ultrasound diagnosis apparatus U according to a second embodiment will now be described.

The ultrasound diagnosis apparatus U according to the second embodiment has configurations identical to those of the first embodiment. These configurations will be referred to by the same reference signs without redundant description.

Operations of the ultrasound diagnosis apparatus U according to the second embodiment in the puncture-needle display mode will now be explained. In the ultrasound diagnosis apparatus U according to the second embodiment, the focal length is not constant along the main scanning direction but rather variable along the traveling direction of the tip of the puncture needle 3. Furthermore, the aperture size (F value) is varied depending on the position of the puncture needle 3, instead of a variable sound-ray density depending on the position of the puncture needle 3.

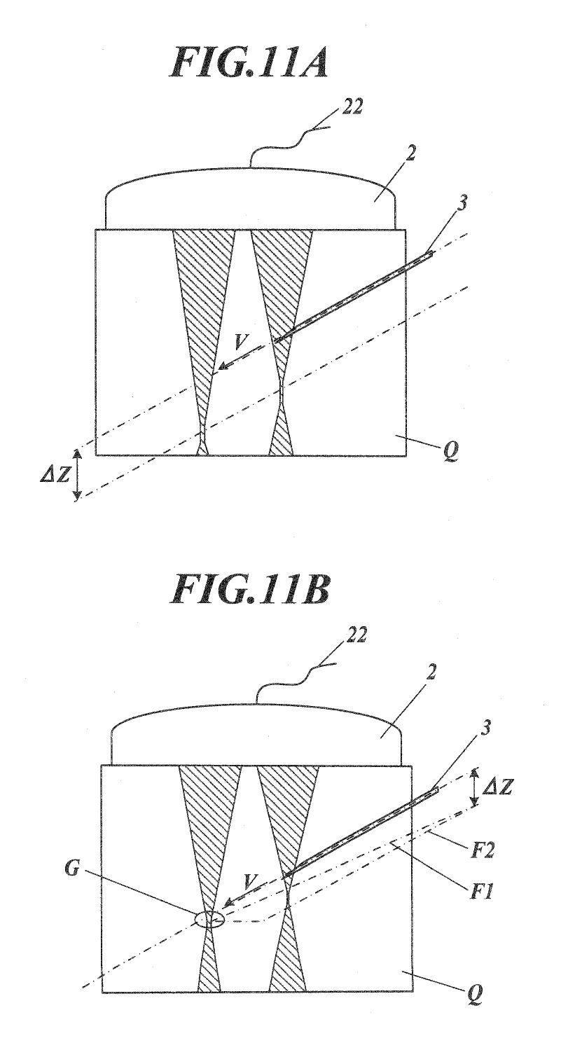

FIGS. 11A and 118 illustrate the setting of the focus position of transmission ultrasounds by the ultrasound diagnosis apparatus U according to the second embodiment.

With reference to FIG. 11A, the ultrasound diagnosis apparatus U according to the second embodiment sets the focal position of ultrasounds transmitted from the oscillators 21 of the ultrasound probe 2 to be displaced from the path of the tip position of the puncture needle 3 and the estimated future path (estimated travel path) to a deeper position (i.e., a position more remote from the source of emission) by the predetermined distance ΔZ in the subject Q, and captures an image of the two-dimensional structure of the subject Q to create a frame image for the B-mode display.

If the target G to receive the puncture needle 3 is identified in the frame image, the displacement (the above-described predetermined distance) of the focal position can be varied depending on the horizontal component of the distance between the tip position of the puncture needle 3 and the target G, as in the distribution F1 of the focal position in FIG. 11B. For example, the focal point may be initially displaced from the tip position of the puncture needle 3 to a deeper position by an initial distance ΔZ in the subject Q, and may approach the target G with a gradual decrease in the distance ΔZ, in response to the approach of the tip position to the target G, to substantially suit the target G. In this case, the focal position may be intentionally displaced from the tip position of the puncture needle 3, immediately before the tip position overlaps with the target G or while the tip position overlaps with the target G, to give priority to receive clear echoes from the puncture needle 3.

Alternatively, the setting of the focal position as illustrated in FIG. 7 and the setting of the distribution of the focal position as illustrated in FIG. 11A may be combined. In specific, the focal position may be at a constant depth equal to the target G or deeper than the target G by a predetermined distance while the difference in depth between the estimated tip position of the puncture needle 3 and the target G is smaller than the initial distance Δ2, as in the distribution F2 of the focal position in FIG. 11B.

The target G may be identified by the automatic detection of the first discontinuity surface in the traveling direction of the puncture needle 3 or by the manual selection by the user. To manually select the target G, the user may touch to designate the target G in a B-mode image on the display screen of the output display 19, or may input data through the keyboard on the basis of the scale displayed in the B-mode image, for example. Alternatively, a region having identical features to the region of the manually selected target G may be automatically detected and followed in future frame images.

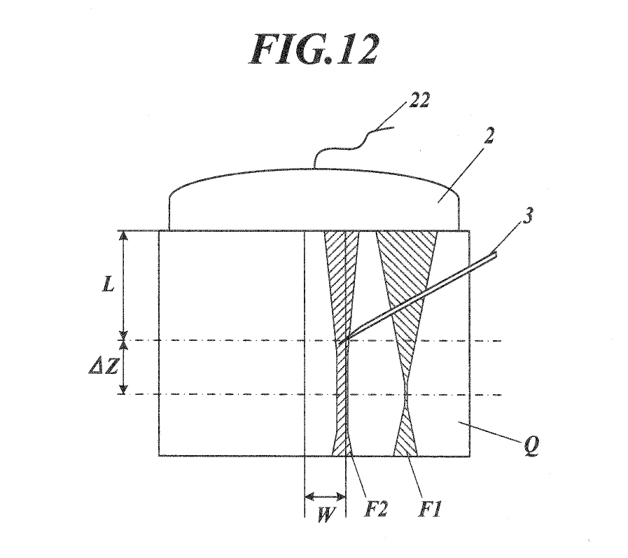

FIG. 12 illustrates the distribution of the aperture sizes of transmission ultrasounds in the cross section along the scanning direction of the ultrasound probe 2.

As in the changing and setting of the sound-ray density illustrated in FIG. 8, the ultrasound diagnosis apparatus U according to the second embodiment defines a predetermined range W (second horizontal range) including the estimated tip position of the puncture needle 3, and sets the aperture size in the predetermined range W to be smaller than the normal size. In other words, the ultrasound diagnosis apparatus U reduces the number N of oscillators 21 used for the capture of a single scanned image in the predetermined range W. This control prevents excess convergence of transmission ultrasound beams in the vicinity of the focal position, and increases the depth of focus, so that both the tip of the puncture needle 3 and a target can be imaged with sufficient focus even if they are deviated from the focal position.

The aperture size can be varied depending on the difference in depth direction between the tip position of the puncture needle 3 and the target. The aperture size may be decreased to increase the depth of focus in response to a large difference in depth between the tip position of the puncture needle 3 and the target, and may be increased with a reduction in the difference in depth between the tip position and the target.

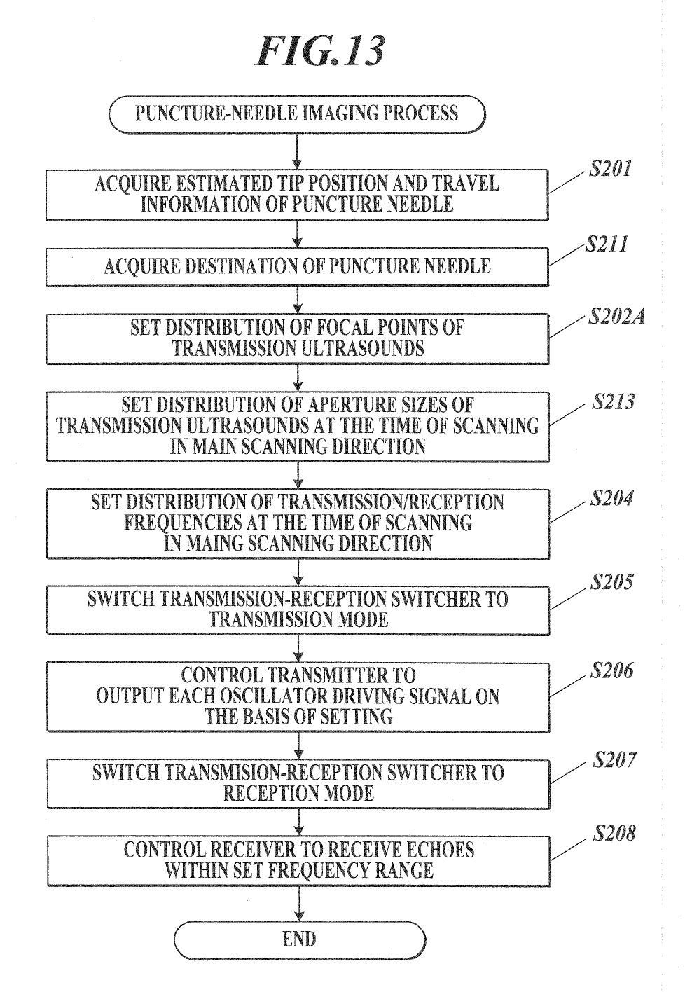

FIG. 13 is an example flowchart illustrating the control procedure of the puncture-needle imaging process executed in the puncture-needle imaging controller 111 in the puncture-needle display mode according to the second embodiment.

The puncture-needle imaging process is identical to that in the first embodiment, except for new Step S211, and Steps S202A and S213 respectively substituted for Steps S202 and S203. The identical steps will be referred to by the same reference signs without redundant description.

The puncture-needle imaging controller 111 (CPU) acquires the estimated tip position of the puncture needle 3 and the traveling information thereof from the image processor 16 (Step S201), and then acquires the destination (the specified position of the target G) of the puncture needle 3 (Step S211). In Step S211, the puncture-needle imaging controller ill may acquire the position of the predetermined range W defined based on the traveling direction of the puncture needle 3 identified by the image processor 16, or may acquire the positional information input from the user through the operation input unit 18.

The puncture-needle imaging controller 111 sets the distribution of the focal positions of transmission ultrasounds (Step S202A). In specific, the puncture-needle imaging controller 111 sets the focal position for each scanning direction on the basis of the estimated tip position and the destination of the puncture needle 3.

The puncture-needle imaging controller 111 also sets the distribution of the aperture sizes of transmission ultrasounds at the time of scanning in the main scanning direction (Step S213). In specific, the puncture-needle imaging controller 111 defines a range on the basis of the traveling direction of the tip position. The range consists of a larger section, which is defined between the tip position and the second (distal) edge of the range in the traveling direction and has a predetermined width corresponding to the traveling speed of the tip position, and a smaller section, which is defined between the tip position and the first (proximal) edge of the range in the opposite direction of the traveling direction. The puncture-needle imaging controller 111 sets the aperture size in the range to be smaller than the normal size.

The puncture-needle imaging controller 111 then proceeds to Step S204.

As described above, in the ultrasound diagnosis apparatus U according to the second embodiment, the puncture-needle imaging controller 111 acquires the calculated value of the estimated travel path of the puncture needle 3 from the image processor 16, and sets the spatial position of the focal point of transmission ultrasounds by displacing the focal point from the estimated travel path by the depth difference within the predetermined range in a scanned area. This control enables the display of the entire future path of the puncture needle 3 to the target with sufficient focus and the clear display of the puncture needle 3.

The puncture-needle imaging controller 111 can displace the focal point of transmission ultrasounds from the estimated travel path of the puncture needle 3 to a deeper position in the subject Q. The focal point is thus determined so that the traffic line of the focal point parallel to the travel path of the puncture needle 3, which enables the stable and certain display with a constant detection level of the puncture needle 3, regardless of some deviation of the tip position of the puncture needle 3 from the estimated position due to a variation in the traveling speed of the puncture needle 3.

The puncture-needle imaging controller 111 can decrease the aperture size (i.e., increase the F value) of transmission ultrasounds in the predetermined range W including the estimated tip position of the puncture needle 3 in comparison with the aperture size (F value) in the external area of the predetermined range W. This control prevents excess convergence of ultrasound beams in the vicinity of the tip of the puncture needle 3 and increases the depth of focus, so that an area of a wide range of depth including the vicinity of the tip can be displayed with sufficient focus. The control thus enables appropriate detection of echoes from the tip of the puncture needle 3 and display of a B-mode image of the two-dimensional structure without largely lowing the resolution.

The predetermined range W for setting the aperture size has the second (distal) edge in the traveling direction of the puncture needle 3 and the first (proximal) edge in the opposite direction in the B-mode image of the two-dimensional structure, and the predetermined range W has a larger width from the estimated tip position to the second edge than the width to the first edge. This configuration can facilitate the high-resolution display of the target G, which is expected to be deviated from the puncture needle 3 in the traveling direction. The configuration can also prevent the deviation of the tip of the puncture needle 3 from the high-resolution display area regardless of a variation in the traveling speed of the puncture needle 3.

### Third Embodiment

The ultrasound diagnosis apparatus U according to a third embodiment will now be described.

The ultrasound diagnosis apparatus U according to the third embodiment has configurations identical to those of the first or second embodiment. These configurations will be referred to by the same reference signs without redundant description.

Operations of the ultrasound diagnosis apparatus U according to the third embodiment in the puncture-needle display mode will now be explained. The ultrasound diagnosis apparatus U according to the third embodiment uses a spatial compounding technique, which involves the reception of multiple patterns (three patterns in this embodiment) of echoes of ultrasounds emitted in different directions and the identification of the two-dimensional structure based on superimposed data on the received data to create a frame image.

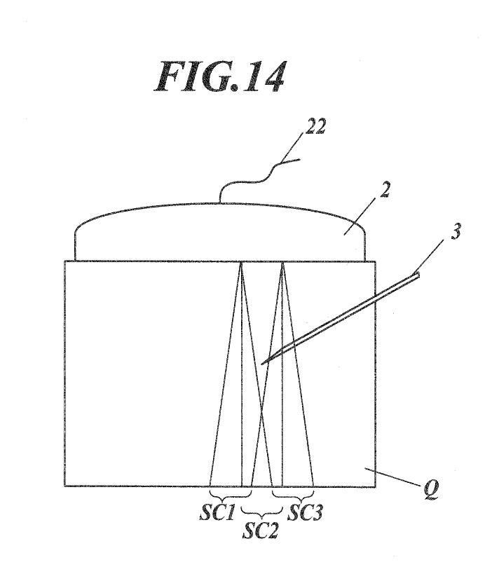

FIG. 14 illustrates the directions of transmission ultrasounds in the cross section along the scanning direction of the ultrasound probe 2.

In this embodiment, ultrasounds are emitted in three different directions to the subject Q by means of the spatial compounding technique. The emission of ultrasounds at multiple angles can provide clear echoes from a target. In specific, even if echoes of the ultrasounds perpendicular to the subject Q have abnormal directivity or include artifacts due to the inclination of the reflecting surface, scattering, or diffraction, or if the ultrasounds are blocked by an obstacle from the target, ultrasounds in other directions can provide clear echoes from the target.

In the ultrasound diagnosis apparatus U according to the third embodiment, at least one of the above-described various settings (i.e., processes of a displacement of the focal position, an increase in the sound-ray density, a decrease in the aperture size, and a reduction of the frequency of transmission/reception ultrasounds) is mainly applied to the ultrasounds transmission at the largest angle from the extending direction of the puncture needle 3 in the transmission ultrasounds in three directions in the spatial compounding technique.

In FIG. 14 illustrating the puncture needle 3 traveling from the upper right to the lower left, at least one of the processes is mainly applied to the ultrasounds SC3 propagating from the upper left to the lower right, whereas no process is applied to the ultrasounds SC1 propagating from the upper right to the lower left. The processes to the ultrasounds SC2 propagating from the top to the bottom in FIG. 14 are optional. An increase in the sound-ray density and a decrease in the aperture size applied to the ultrasounds SC2 may be respectively smaller than those applied to the ultrasounds SC3. Alternatively, the number of the processes applied to the ultrasounds SC2 may be smaller than that of the ultrasounds SC3.

As described above, the ultrasound diagnosis apparatus U according to the third embodiment can create a two-dimensional image on the basis of echoes of ultrasounds transmitted in different directions from the ultrasound probe 2 to the subject Q by means of the spatial compounding technique, and can provide the display in B-mode. In this case, the puncture-needle imaging controller 111 can adjust the focus position, the sound-ray density, and the aperture size of ultrasounds, such that variations in these parameters of the ultrasounds to be transmitted/received at a larger angle from the traveling direction of the puncture needle 3 are respectively greater than those of the ultrasounds to be transmitted/received at a smaller angle, in the B-mode image of the two-dimensional structure. In other words, the adjustments of the parameters according to the invention are applied to the ultrasounds in a certain direction that are readily reflected from the tip of the puncture needle 3. This control enables the efficient and certain detection of echoes from the tip of the puncture needle 3 and the display of the tip.

The above embodiments should not be construed to limit the invention, and may be modified in various manners.

For example, although the ultrasound probe 2 performs scanning in a predetermined single direction to capture and display a B-mode image in the embodiments, the ultrasound probe 2 may be capable of two-dimensional scanning. In this case, the focal position, sound-ray density, aperture size, and frequency band of ultrasounds in the two directions may be individually controlled to detect the tip of the puncture needle 3 only within a required area.

The first embodiment uses the combination of the control of the focal position and the sound-ray density and the frequency compounding technique, the second embodiment uses the combination of the setting of the spatial distribution of the focal position and the control of the aperture size, whereas the third embodiment uses the spatial compounding technique. Alternatively, these techniques may be used alone, or in combination within an executable range.

The parameters, such as the focal position, may be set based on the estimated tip position of the puncture needle 3 alone or in combination with the position of the target G at the beginning of the display, and may be set based on the estimated travel path of the puncture needle 3 after a certain accumulation of the history of the tip position of the puncture needle 3.

Although the focal position is positioned deeper than the estimated tip position of the puncture needle 3 in the embodiments, the focal position may be positioned shallower than the estimated tip position. Any pattern of spatial or temporal displacement of the focal position other than the above-described examples may be employed provided that the focal position is displaced from the estimated tip position of the puncture needle 3.

Although the predetermined range W has a larger width in the traveling direction of the puncture needle 3 in the embodiments, this configuration should not be construed to limit the invention. The predetermined range W may be defined based on the previously identified tip position instead of the estimated tip position. Such a predetermined range W including the previous tip position certainly includes the tip position regardless of a temporary stop of the puncture needle 3.

Different predetermined ranges W may be defined for the respective adjusting processes of the sound-ray density, reception frequency band, and aperture size of ultrasounds. For example, the increase range of the sound-ray density may be set in an appropriate range below the predetermined upper limit depending on the correlation between the decrease range of the sound-ray density and the frame rate to be not changed. The reception frequency band may be shifted in a wider range.

Although the traveling direction of the puncture needle 3 and the scanning direction of the ultrasound probe 2 reside in the same plane in the embodiments, they do not need to be substantially in the same plane. In other words, the traveling direction may be inclined from the scanning direction provided that the tip position of the puncture needle 3 can be detected. Alternatively, the traveling direction of the puncture needle 3 may be vertical or horizontal instead of being diagonal in a B-mode image of the two-dimensional structure.

Although the sound-ray densities in the predetermined range W and its external area are increased and decreased, respectively, to maintain the frame rate in the embodiments, this configuration should not be construed to limit the invention. For example, the frame rate may be decreased while not decreasing the sound-ray density in the external area.

Although the tip position of the puncture needle 3 is identified based on frame images or the operation information of the attachment 4 in the embodiments, this configuration should not be construed to limit the invention. For example, the tip position of the puncture needle 3 may be identified through the reception and measurement of electric waves, electric fields, or magnetic fields from the tip and/or multiple middle points (e.g., two middle points) of the puncture needle 3.

The identification of the tip position of the puncture needle 3 and the estimation of the current tip position based on the identified tip position do not necessarily follow every creation of a frame image. For a high frame rate relative to the traveling speed of the puncture needle 3, these operations may be executed after every creation of a predetermined number of frame images, for example, depending on the load on the CPU during the identification of the tip position.

Any specific configuration in the embodiments may be appropriately modified within the gist of the invention.

The present U.S. patent application claims a priority under the Paris Convention of Japanese patent application No. 2014-112487 filed on May 30, 2014, in which all contents of this application are disclosed, and which shall be a basis of correction of an incorrect translation.

What is claimed is:

1. An ultrasound diagnosis apparatus comprising:
a transmitter/receiver that transmits at least one ultrasound to a subject, and receives a reflected wave of the ultrasound from the subject;
a processor that processes data on the received reflected wave to create at least one image of a two-dimensional structure including a depth direction of the subject;
a display controller that controls a display to display the created image of the two-dimensional structure;
a position acquirer that acquires an estimated tip position of a puncture needle to be stuck into the subject; and
a transmission controller that adjusts at least one parameter of a focal length, aperture size and sound-ray density of the ultrasound depending on the acquired estimated tip position, and controls the transmitter/receiver to transmit the ultrasound.

2. An ultrasound diagnosis apparatus comprising:
a transmitter/receiver that transmits an ultrasound to a subject, and receives a reflected wave of the ultrasound from the subject;
a processor that processes data on the received reflected wave to create an image of a two-dimensional structure including a depth direction of the subject;
a frequency setter that sets at least one of a frequency band of the ultrasound used for creating the image of the two-dimensional structure and an intensity distribution in the frequency band;
a display controller that controls a display to display the created image of the two-dimensional structure; and
a position acquirer that acquires an estimated tip position of a puncture needle to be stuck into the subject, wherein
the frequency setter executes at least one of settings: to shift the frequency band used for creating the image of the two-dimensional structure to a lower frequency side within a predetermined range including the acquired estimated tip position in comparison with the frequency band in an external area of the predetermined range; and to shift the intensity distribution to a lower frequency side of the frequency band within the predetermined range in comparison with the intensity distribution in the external area.

3. The ultrasound diagnosis apparatus of claim 1, wherein the transmission controller displaces a focal position of the transmitted ultrasound from the estimated tip position.

4. The ultrasound diagnosis apparatus of claim 3, wherein the transmission controller varies the focal length according to a shift of the estimated tip position.

5. The ultrasound diagnosis apparatus of claim 3, wherein the transmission controller sets the focal position of the transmitted ultrasound so that a depth difference between the focal position and the estimated tip position in the subject is within a predetermined range.

6. The ultrasound diagnosis apparatus of claim 5, wherein the transmission controller determines the depth difference within the predetermined range based on a distance or a difference in depth in the subject between the estimated tip position and a destination of the puncture needle.

7. The ultrasound diagnosis apparatus of claim 3, wherein the transmission controller sets the focal position of the transmitted ultrasound to a deeper position than the estimated tip position in depth in the subject.

8. The ultrasound diagnosis apparatus of claim 3, wherein
the position acquirer acquires an estimated travel path of the puncture needle, and
the transmission controller displaces the focal position of the transmitted ultrasound from the estimated travel path of the puncture needle so that a depth difference between the focal position and the estimated travel path is within a predetermined range.

9. The ultrasound diagnosis apparatus of claim 8, wherein the transmission controller sets the focal position to a deeper position than the estimated travel path of the puncture needle in depth in the subject.

10. The ultrasound diagnosis apparatus of claim 3, wherein the transmission controller increases the sound-ray density of the transmitted ultrasound in a first horizontal range including the estimated tip position in comparison with the sound-ray density in an external area of the first horizontal range.

11. The ultrasound diagnosis apparatus of claim 10, wherein the first horizontal range has a first edge and a second edge, and has a larger width from the estimated tip position to the second edge in a traveling direction of the puncture needle than a width from the estimated tip position to the first edge in the opposite direction, in a plane of the two-dimensional structure.

12. The ultrasound diagnosis apparatus of claim 10, wherein the transmission controller decreases the sound-ray density in the external area of the first horizontal range correspondingly to an increase in the number of transmission of the ultrasound caused by the increased sound-ray density in the first horizontal range so that the number of transmission of the ultrasound in the external area is decreased.

13. The ultrasound diagnosis apparatus of claim 3, wherein the transmission controller decreases the aperture size of the transmitted ultrasound in a second horizontal range including the estimated tip position in comparison with the aperture size in an external area of the second horizontal range.

14. The ultrasound diagnosis apparatus of claim 13, wherein the second horizontal range has a first edge and a second edge, and has a larger width from the estimated tip position to the second edge in a traveling direction of the puncture needle than a width from the estimated tip position to the first edge in the opposite direction, in a plane of the two-dimensional structure.

15. The ultrasound diagnosis apparatus of claim 1, wherein
the processor creates the image of the two-dimensional structure based on reflected waves of the ultrasounds transmitted in different directions from the transmitter/receiver to the subject, and
the transmission controller controls a variation in the adjusted parameter of the ultrasound to be transmitted/received at a larger angle from a traveling direction of the puncture needle to be greater than a variation in the adjusted parameter of the ultrasound to be transmitted/received at a smaller angle from the traveling direction, in a plane of the two-dimensional structure.

16. The ultrasound diagnosis apparatus of claim 1, further comprising:
a frequency setter that sets at least one of a frequency band of the ultrasound used for creating the image of the two-dimensional structure and an intensity distribution in the frequency band, wherein
the frequency setter executes at least one of settings: to shift the frequency band used for creating the image of the two-dimensional structure to a lower frequency side within a predetermined range including the acquired estimated tip position in comparison with the frequency band in an external area of the predetermined range; and to shift the intensity distribution to a lower frequency side of the frequency band within the predetermined range in comparison with the intensity distribution in the external area.

17. The ultrasound diagnosis apparatus of claim 1, wherein the display controller controls the display to display a predetermined indication when the transmitter/receiver transmits/receives the ultrasound whose parameter is adjusted by the transmission controller.

18. The ultrasound diagnosis apparatus of claim 2, wherein the display controller controls the display to display a predetermined indication when the transmitter/receiver receives the ultrasound including at least one of the frequency band and the intensity distribution in the frequency band which are shifted to the lower frequency side by the frequency setter.

19. The ultrasound diagnosis apparatus of claim 1, wherein the position acquirer acquires the estimated tip position based on a difference among the multiple images of the two dimensional structures acquired at different timings.

20. The ultrasound diagnosis apparatus of claim 1, wherein the position acquirer acquires the estimated tip position based on information on a traveling length and direction of the puncture needle, the information being obtained from a puncturing mechanism of the puncture needle.
